(19) United States
(12) Patent Application Publication
(10) Pub. No.: US 2025/0000931 A1
(43) Pub. Date: Jan. 2, 2025

(54) PHARMACEUTICAL COMPOSITION FOR PREVENTING OR TREATING PAIN, COMPRISING GR82334 AS ACTIVE INGREDIENT

(71) Applicant: KOREA UNIVERSITY RESEARCH AND BUSINESS FOUNDATION, Seoul (KR)

(72) Inventors: June Sun KIM, Seoul (KR); Min Ji KWON, Siheung-si (KR); Hea Yeon YOO, Seoul (KR)

(73) Assignee: KOREA UNIVERSITY RESEARCH AND BUSINESS FOUNDATION, Seoul (KR)

(21) Appl. No.: 18/707,072

(22) PCT Filed: Oct. 20, 2022
(86) PCT No.: PCT/KR2022/016032

§ 371 Date: May 2, 2024

(30) Foreign Application Priority Data
Nov. 3, 2021 (KR) 10-2021-0149854

(51) Int. Cl. A61K 38/08; A61P 25/04; A61P 29/00

(52) U.S. Cl. CPC A61K 38/08; A61P 25/04; A61P 29/00

(57) ABSTRACT

The present invention relates to a pharmaceutical composition for preventing or treating pain, comprising GR82334 as an active ingredient. Particularly, the present invention relates to: a pharmaceutical composition for preventing or treating pain, comprising GR82334 as an active ingredient; and a pain treatment method comprising a step of intra-articularly or intrathecally administering the pharmaceutical composition of the present invention to an individual in which pain arises.

### TECHNICAL FIELD

The present invention relates to a pharmaceutical composition for preventing or treating pain containing GR82334 as an active ingredient.

### BACKGROUND ART

Pain affects millions of those who suffer from diseases such as arthritis, cancer and diabetes after injury or surgery. Pain may be classified in consideration of the location of occurrence, cause, characteristic, and mechanism of occurrence.

Pain may be divided into acute pain and chronic pain depending on the duration thereof and typical chronic pain diseases include neuropathic pain, complex regional pain syndrome, post-herpetic neuralgia, and pain syndrome after spinal surgery. Chronic pain is not simply an extension of acute pain; the degree of pain is not proportional to the extent of the original damage and may develop spontaneously without stimulation. When the duration and intensity of pain cause detrimental effects on the functions or life of patients or persists beyond the normal tissue healing period, generally 3 months, this pain is defined as chronic pain and is considered a disease rather than a symptom.

Chronic pain is unremitting, is not self-controlled, and may persist for years and even decades after the initial injury. Chronic pain is primarily neuropathic in nature and may involve peripheral or central nervous system damage.

Among the various types of pain, neuropathic pain refers to pain that occurs even though there is no apparent cause of the pain or injury. Neuropathic pain is a general term for pain caused by damage to nerve cells or abnormalities in the nervous system, such as nerve compression or nerve trauma caused by cancer, autoimmune disease, or herpes zoster infection. Neuropathic pain is chronic pain that shows clinical characteristics such as allodynia, which is a painful response to stimuli that would not cause pain in a normal state, hyperalgesia which has a more sensitive response to painful stimuli, and spontaneous burning sensation even in the absence of stimulation. Unlike acute pain, which has the advantage of protecting the body from harmful stimuli, such neuropathic pain causes pain beyond the disease due to deformation of the nervous system that transmits pain.

Pain relievers currently used to treat pain, that is, analgesics, are mainly anti-inflammatory drugs such as aspirin or Tylenol, and morphine-based drugs are used for severe pain. In addition, ketoprofen and capsaicin are available in the form of creams or patches. However, these commercially available pain relievers do not have great pharmacological effects and cause side effects. Excessive doses of aspirin and Tylenol may cause toxicity to the stomach and liver, causing disability and allergies. In addition, opioid painkillers such as morphine may cause strong addiction in patients, and non-steroidal anti-inflammatory drugs (NSAIDs) have various undesirable side effects such as nausea, vomiting, constipation, and blood coagulation. Therefore, there is a need for development of novel pain treatments that have fewer or no side effects and exhibit excellent effects of relieving and treating pain.

Meanwhile, GR82334 is known to be an antagonist of the NK1 receptor, but the potential use thereof as a pain therapeutic agent has not been known.

### DISCLOSURE

### Technical Problem

Accordingly, the present inventors found that GR82334 can relieve and ameliorate pain, while controlling inflammation, and thus has the potential to be used as a novel therapeutic agent for pain. Based thereon, the present invention has been completed.

It is one object of the present invention to provide a pharmaceutical composition for preventing or treating pain containing GR82334 as an active ingredient.

It is another object of the present invention to provide a method of treating pain including intra-articularly or intrathecally administering the composition of the present invention to a subject suffering from pain.

### Technical Solution

In accordance with an aspect of the present invention, the above and other objects can be accomplished by the provision of a pharmaceutical composition for preventing or treating pain containing GR82334 as an active ingredient.

In one embodiment of the present invention, the composition may be administered intra-articularly or intrathecally.

In one embodiment of the present invention, the pain may be chronic pain.

In one embodiment of the present invention, the chronic pain may be selected from the group consisting of neuropathic pain, complex regional pain syndrome, postherpetic neuralgia, and pain syndrome after spinal surgery.

In one embodiment of the present invention, the neuropathic pain may be induced by at least one nerve damage selected from the group consisting of peripheral nerve damage, central nerve damage, and inflammatory nerve damage.

In one embodiment of the present invention, the neuropathic pain may be selected from the group consisting of multiple sclerosis including abnormalities of or damage to the peripheral nervous system and spinal nerve damage, spinal cord injury, hyperalgesia, hyperesthesia, neuropathy, diabetic neuropathy, neuritis, neuralgia, burning pain, allodynia, postherpetic neuralgia, lumbar nerve root compression, cancer-related pain, pain due to alcoholism, atypical facial pain, herpetic neuralgia, poststroke pain, HIV-related neuropathy, osteoarthritis pain, joint pain, and phantom limb pain.

In one embodiment of the present invention, GR82334 may relieve or reduce pain through activity of blocking the SP (substance P)/NK1 (neurokinin 1) signaling pathway.

In one embodiment of the present invention, GR82334 may reduce expression of inflammatory cytokines and increase expression of anti-inflammatory cytokines.

In one embodiment of the present invention, the inflammatory cytokines may be COX-2 and IL-1β, and the anti-inflammatory cytokines may be IL-6.

In accordance with another aspect of the present invention, provided is a method of treating pain including intra-articularly or intrathecally administering the composition of the present invention to a subject suffering from pain.

### Advantageous effects

GR82334 according to the present invention can suppress inflammatory reactions and simultaneously suppress the expression of pain-related signaling substances by blocking the SP/NK1 signaling pathway, which transmits pain, thus being useful as a novel potent therapeutic agent for pain.

### DESCRIPTION OF DRAWINGS

FIG. 1 shows the result of analysis of the analgesic effect after intra-articular administration of GR82334 (10 μM/30 μl) to rats (white papers) with induced chronic joint pain, wherein (A) shows the result of PWT measurement over time after drug administration, (B) shows the result of MPE (maximal possible effect) analysis based on PWT (A), and (C) shows the result of AUC (area under the curve) analysis based on MPE (B).

FIG. 2 shows the result of analysis of the analgesic effect after intrathecal administration of GR82334 (10 μM/10 μl) to rats (white papers) with induced chronic joint pain, wherein (A) shows the result of PWT measurement over time after drug administration, (B) shows the result of MPE (maximal possible effect) analysis based on PWT (A), and (C) shows the result of AUC (area under the curve) analysis based on MPE (B).

FIG. 3 shows the result of analysis of the analgesic effect after intra-articular administration of GR82334 at different concentrations (10 μM, 1 μM, and 100 nM/30 μl) to rats (white papers) with induced chronic joint pain, wherein (A) shows the result of PWT measurement over time after drug administration, (B) shows the result of MPE (maximal possible effect) analysis based on PWT (A), and (C) shows the result of AUC (area under the curve) analysis based on MPE (B).

FIG. 4 shows the result of analysis of the analgesic effect after intrathecal administration of GR82334 at different concentrations (10 μM, 1 μM, and 100 nM/30 μl) to rats (white papers) with induced chronic joint pain, wherein (A) shows the result of PWT measurement over time after drug administration, (B) shows the result of MPE (maximal possible effect) analysis based on PWT (A), and (C) shows the result of AUC (area under the curve) analysis based on MPE (B).

FIG. 5 shows the results of expression levels of CGRP, SP, and NK1 in the knee joint by immunofluorescence after intra-articularadministration of GR82334 to rats (or white papers)) with induced chronic joint pain in an embodiment of the present invention.

FIG. 6 shows the results of expression levels of CGRP, SP, and NK1 in the knee joint by immunofluorescence after intrathecal administration of GR82334 to rats (or white papers) with induced chronic joint pain in an embodiment of the present invention.

FIG. 7 shows the expression levels of pain-related neuropeptides (SP, CGRP) receptors (NK1R, TRPV1), and inflammatory factors in the spinal dorsal horn or ipsilateral dorsal quadrants of the spinal segment after intra-articular administration of GR82334 to rats (or white papers) with induced chronic joint pain in an embodiment of the present invention, wherein (A) shows analysis results of SP expression in the spinal doral horn through DAB analysis, (B) shows analysis results of CGRP expression in the spinal dorsal horn through DAB analysis, (C) shows the results of quantitative analysis of protein expression of NK1R, (D) shows the results of quantitative analysis of protein expression of TRPV1, (E) shows the results of quantitative analysis of protein expression of COX2, (F) shows the results of quantitative analysis of protein expression of IL-1beta, (G) shows the results of quantitative analysis of protein expression of IL-6, and (H) shows the results of protein band of NK1R and TRPV1 in the ipsilateral dorsal quadrants of spinal segment (L3-5) through western blot.

FIG. 8 shows the expression levels of pain-related neuropeptides (SP, CGRP) and receptors (NK1R, TRPV1), and inflammatory cytokines in the spinal dorsal horn or ipsilateral dorsal quadrants of spinal segment after intrathecal administration of GR82334 to rats (or white papers) with induced chronic joint pain in an embodiment of the present invention, wherein (A) shows analysis results of SP expression in the spinal dorsal horn through DAB analysis, (B) shows analysis results of CGRP expression in the spinal dorsal horn through DAB analysis, (C) shows the results of quantitative analysis of protein expression of NK1R, (D) shows the results of quantitative analysis of protein expression of TRPV1, (E) shows the results of quantitative analysis of protein expression of COX2, (F) shows the results of quantitative analysis of protein expression of IL-1beta, (G) shows the results of quantitative analysis of protein expression of IL-6, and (H) shows the results of protein band of NK1R and TRPV1 in the ipsilateral dorsal quadrants of spinal segment (L35) through western blot.

FIG. 9 shows a process of measuring PWT (paw withdrawal threshold) to graded mechanical stimulus with a series of von Frey filaments in rats.

FIG. 10 shows a method of intra-articularly administering the drug to rats (white rats or white papers).

FIG. 11 shows a method of intrathecally administering the drug to rats (white rats or white papers).

### BEST MODE

The present invention provides a novel pharmaceutical use of GR82334, i.e., the possibility of the use of GR82334 as a therapeutic agent for pain.

Specifically, the present invention provides a pharmaceutical composition for preventing or treating pain containing GR82334 as an active ingredient.

In the present invention, GR82334 is known as an antagonist of NK1 (neurokinin 1) receptor having the following Formula and no results have been reported showing its efficacy in treating pain.

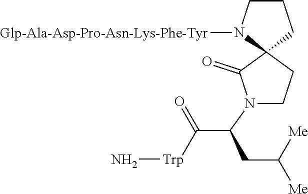

### Formula of GR82334 Compound

Meanwhile, the present inventors confirmed through experiments the possibility of using GR82334 as a novel therapeutic agent for pain that can effectively control pain while reducing side effects.

In relation to this, in one embodiment of the present invention, GR82334 is administered to rats (or white paper) with induced chronic joint pain, that is, rats (or white paper) in the actual chronic pain maintenance stage after inducing arthritis. Then, the degree of pain relief was analyzed. The result shows that the group administered GR82334 is more effective in relieving pain than the control group administered a saline solution.

In addition, in another embodiment of the present invention, the degree of pain relief depending on the route of administration of GR82334 was analyzed. Rats (rats or white papers) with chronic joint pain were divided into a group administered intra-articularly the drug and a group administered intrathecally the drug. The result of analysis on the degree of pain relief in the drug-administered groups shows that intrathecal administration could effectively relieve pain even with a lower concentration of drug compared to intra-articular administration.

In another embodiment of the present invention, in order to determine the pain-relief mechanism of action of GR82334, after administering GR82334 to rats (white papers) with chronic joint pain, changes in expression of the neuropeptides CGRP (calcitonin gene-related peptide), SP (substance P), and NK1 (neurokinin 1) receptors were analyzed.

The result shows that the expression of neuropeptides involved in pain transmission was found to be reduced in the GR82334-administered group compared to the control group.

In addition, the GR82334-administered group exhibited a decrease in the inflammatory cytokines COX-2 and IL-1β compared to the control group, but an increase in the expression level of the anti-inflammatory cytokine IL-6.

These results show that the drug GR82334 has effects of blocking SP-NK1 signaling, having an anti-inflammatory effect and reducing pain signals, thus effectively preventing or treating pain.

Therefore, the present invention provides a pharmaceutical composition for preventing or treating pain containing GR82334 as an active ingredient.

In the present invention, the pain may be chronic pain. The term “chronic pain disease” refers to moderately severe pain that affects one or more sites of the body and lasts for more than three months, and includes pain syndromes, the intensity of which may vary over time.

The chronic pain includes, but is not limited to, neuropathic pain, complex regional pain syndrome, postherpetic neuralgia, or pain syndrome after spinal surgery.

The term “neuropathic pain” may be induced by peripheral nerve damage, central nerve damage, or inflammatory nerve damage. Specifically, the neuropathic pain includes, but is not limited to, multiple sclerosis including abnormalities of or damage to the peripheral nervous system and spinal nerve damage, spinal cord injury, hyperalgesia, hyperesthesia, neuropathy, diabetic neuropathy, neuritis, neuralgia, burning pain, allodynia, postherpetic neuralgia, lumbar nerve root compression, cancer-related pain, pain due to alcoholism, atypical facial pain, herpetic neuralgia, poststroke pain, HIV-related neuropathy, osteoarthritis pain, joint pain, and phantom limb pain.

The target disease that can be treated by the composition of the present invention may include all of the pain diseases described above. In one embodiment of the present invention, the therapeutic efficacy of GR82334 administration was determined in a rat (or white paper) animal model caused by chronic joint pain, which is a type of chronic pain and corresponds to neuropathic pain caused by neurogenic inflammation.

In particular, in one embodiment of the present invention, GR82334 administration exhibits the activity of blocking the signal of SP-NK1, which is one of the main mechanisms of chronic pain caused by neuroplastic changes after arthritis.

SP (Substance P) is secreted from sensory nerves to the periphery and spinal cord, causing neurogenic inflammation and thus a chronic inflammatory response, and activating the NK1 receptor (neurokinin 1 receptor) involved in pain transmission, thereby increasing the activity of pain-transmitting nerve fibers. These sequence action finally induces hyperalgesia caused by central sensitization.

Therefore, SP (substance P) is an important factor involved in the initial inflammatory response and pain generation, in neurogenic inflammation that causes a vicious cycle of inflammation and pain, and in increased excitability of the central nervous system, which is a major mechanism of chronic pain.

Damaged peripheral nerves secrete substances such as glutamate, CGRP (calcitonin gene-related peptide), and SP (substance P). These substances increase the excitability of the spinal cord to harmful stimuli and secrete neurotransmitters or neuropeptides interfering the function of surrounding inhibitory interneurons. The secreted substances break the pain control balance and cause loss of control over pain.

In addition, it has been reported that continuous inflammatory response in joints where arthritis is induced and SP expression in the joint synovial membrane are increased. Therefore, the mechanism of incurable chronic joint pain may be central sensitization of the spinal cord, due to peripheral nociceptive sensitization and neurogenic.

In addition, it was considered that the inflammatory response had no correlation with pain, especially neuropathic pain, but it has recently been known that the inflammatory response in the peripheral nerves plays an important role in the occurrence of neuropathic pain. Therefore, inflammation should be controlled along with pain in order to effectively control pain.

Therefore, in order to effectively treat chronic pain, it is necessary to suppress the inflammatory response and block pain nerve signaling which is new penetrated and pain signal transmission.

In this regard, it was identified that GR82334 of the present invention has SP/NK1 signal blocking activity and can suppress responses through this inflammatory activity and suppress the expression of pain signaling substances, thus providing fundamental and effective pain treatment.

The composition provided by the present invention contains a pharmaceutically acceptable carrier. The pharmaceutically acceptable carrier that is contained in the pharmaceutical composition of the present invention includes carriers commonly used in preparation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, saline solution, PBS (phosphate buffered saline) or media, but is not limited thereto.

As used herein, the term “pharmaceutically acceptable” refers to a composition that is physiologically acceptable and does not typically cause allergic or similar reactions when administered to humans.

In addition to the above ingredients, the pharmaceutical composition of the present invention may further contain lubricants, wetting agents, sweeteners, flavoring agents, emulsifiers, suspending agents, preservatives, and the like. Suitable pharmaceutically acceptable carriers and preparations are described in the following literature (Remington's Pharmaceutical Sciences, 19th ed., Mack Publishing Company, Easton, PA, 1995).

The preparations for parenteral administration may be formulated as injections using methods known in the art. These formulations are described in the literature (Remington's Pharmaceutical Science, 15^{th} Edition, 1975.Mack Publishing Easton, Company, Pennsylvania 18042, Chapter 87: Blaug, Seymour), which is generally known in the pharmaceutical chemistry.

The pharmaceutical composition of the present invention may be administered orally or parenterally, and may be, for example, administered intrathecally, intravenously, intramuscularly, intraarterially, intramedullary, intrathecally, intracardially, transdermally, subcutaneously, intraperitoneally, intranasally, enterally, topically, sublingually or rectally, and preferably administered intra-articularly or intrathecally. The pharmaceutical composition may be used simultaneously, or sequentially in combination with another ingredient for pain treatment.

The term “intrathecal administration” refers to administering the pharmaceutical composition of the present invention to the spinal cord such that the active ingredient contained in the pharmaceutical composition for preventing or treating chronic pain diseases such as neuropathic pain is delivered into the spinal cord.

In addition, the composition of the present invention is administered in a pharmaceutically effective amount. As used herein, the term “pharmaceutically effective amount” refers to an amount sufficient to treat a disease, and the effective dose may vary depending on factors including the severity of the disease, the age, weight, health, and gender of the patient, the sensitivity of the patient to the drug, time of administration, route of administration and rate of excretion, duration of treatment, and the composition of the present invention used and the drugs combined or used simultaneously, and other factors known in the medical field. Preferably, the effective amount of the pharmaceutical composition of the present invention may vary depending on the degree of the disease, but is preferably administered in an effective amount of 150 to 5,000 μg/60 kg/day, more preferably 500 to 1,500 μg/60 kg/day several times a day. The dose of the composition does not limit the scope of the present invention in any way.

The pharmaceutical composition of the present invention may be administered to mammals, for example, mammals including humans.

The present invention provides a method of treating pain including administering GR82334 to a subject.

In this case, the administration may be administration intra-articularly or intrathecally.

The subject includes, but is not limited to, canines, felines, boars, bovines, deer, giraffes, peccaries, camelids, hippopotamuses, equines, tapirs, rhinoceroses, weasels, lagomorphs, rodents, and primates.

### MODE FOR INVENTION

Hereinafter, the present invention will be described in detail with reference to Examples. The following Examples are provided only for illustration of the present invention and should not be construed as limiting the scope of the present invention.

### Preparation Example and Experiment Method

### \textcircled{1} Preparation of Experimental Animals

The animals used in Examples of the present invention were Sprague-Dawley (SD) rats and the animals could access ad libitum to water and food and were bred under a 12-hour light/12-hour dark cycle. All behavioral tests, including animal care and pain testing, were conducted according to the ethical guidelines of the Korea University Institutional Animal Care and Use Committee.

### \textcircled{2} Modeling of Animal with Osteoarthritis Induced-Chronic Pain through MIA (Monosodium Iodoacetate) Injection

The MIA-induced arthritis model is one of osteoarthritis models. MIA is known to inhibit the activity of glyceraldehyde-3 phosphate dehydrogenase in cartilage cells within the joint, thereby suppressing glycolysis, inducing apoptosis, and degenerating cartilage. Accordingly, the present inventors anesthetized Sprague-Dawley rats (SD rats) weighing 180 to 200 g by inhalation of mixed anesthetic gas (3% isoflurane+97% oxygen) using a mask and confirmed anesthesia using pedal reflex. Then, osteoarthritis was induced by injecting MIA (4 mg of MIA in 50 μl saline solution, Sigma, St Louis, MO, USA) into the right knee joint space using a 30 G insulin syringe.

### \textcircled{3} Response Analysis to Stimulation (von Frey Filament Test)

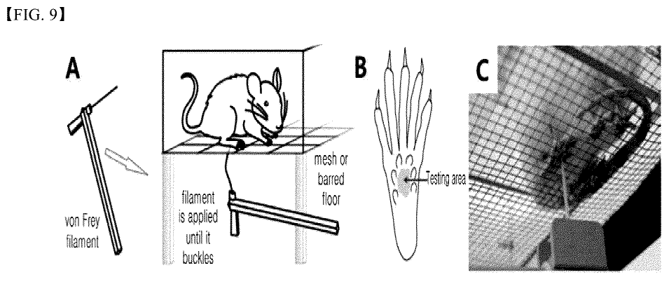

As a method of evaluating the analgesic effect after inducing arthritis and injection of GR82334, the paw withdrawal threshold to the von Frey filament was measured using the up/down method to quantify the mechanical sensitivity of the paw (see FIG. 9). To measure sensory changes in the soles of the paw, rats were placed in a transparent cage (10×10×28 cm) on a stainless steel mesh and were allowed to be without any treatment until they were acclimated to the new environment (about 20 minutes). After the rats were fully acclimated to the new environment, paw withdrawal threshold was measured using eight von Frey filaments (0.4, 0.6, 1, 2, 4, 6, 8, 15 g; Stoelting, Wood Dale, IL, USA). Active paw lift in response to von Frey application was considered an avoidance response. The first stimulus was 2 g and the next weakest filament was applied when there was an avoidance response. When there was no response, the next strongest filament was applied. Interpolation at a 50% threshold was determined in accordance with the method of Dixon (1980).

In addition, sensory changes in the soles of the paw were tested over time before and after induction of arthritis and before and after drug administration. Since the test was based on the avoidance ability of the lower extremities, the test of motor function must be performed and considered during analysis. Normal rats exhibited almost no avoidance response, but animals that suffer from pain due to mechanical stimulation exhibited a rapid avoidance response, which was used as an indicator of mechanical allodynia.

To evaluate drug efficacy, the maximum possible effect (MPE) and the area under the curve (AUC) of MPE were calculated using the measured mechanical allodynia values.

### \textcircled{4} Preparation for GR82334

1 ml of distilled water was added to 1 mg of GR82334 (Tocris, 1670, Avonmouth, Bristol, UK) used in this experiment in accordance with the manufacturer's protocol to prepare a 1 mg/ml stock solution and then the stock solution was diluted at a desired concentration in saline solution, which was used for injection.

### \textcircled{5} After Induction of Arthritis, Intra-Articular Injection of GR82334

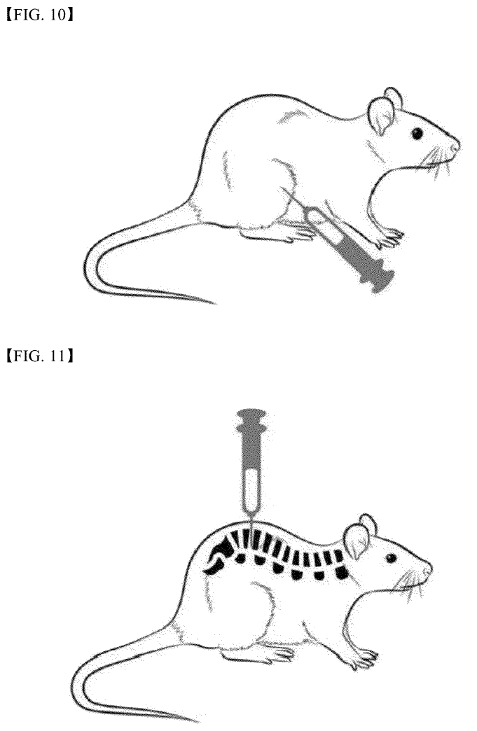

The occurrence of pain was identified through PWT (PWT<1 g) and then drug administration was performed. To inject the drug into the joint cavity, inhalation anesthesia (confirmation of anesthesia with pedal reflex) using a mixed anesthetic gas (3% isoflurane+97% oxygen) was performed using a mask and GR82334 was injected into the right knee joint cavity using a 30 G insulin syringe (see FIG. 10).

### \textcircled{6} After Inducing Arthritis, Intrathecal Injection of GR82334

The occurrence of pain was identified through PWT (PWT<1 g) and then drug administration was performed. To inject the drug intrathecally, inhalation anesthesia (confirmation of anesthesia with pedal reflex) using a mixed anesthetic gas (3% isoflurane+97% oxygen) was performed using a mask. At this time, a hot pad was turned on to maintain body temperature during the procedure. After anesthesia, the hair was removed from the treatment site, and local disinfection was performed three times using 70% ethanol and a povidone-iodine cotton ball. After removing the muscles around lumbar vertebrae 4-5 (L4-5) using fine scissors, the muscles and tendons between the spines were removed using a rongeurs. An 18 G needle was finely inserted between lumbar vertebrae L4 and L5 as a guide needle, and then PE10 tube was inserted approximately 3 cm through the needle. GR82334 was injected into the opposite end of the inserted PE10 tube using a 30 G insulin syringe. Then, the PE10 tube and needle was removed, and the muscle and skin were sutured using 3-0 silk.

### \textcircled{7} After Inducing Arthritis, Tissue Extraction and Histological Examination 9 Hours and 2 Days after Intra-Articular and Intrathecal Injection of GR82334

Immunofluorescence: A mixture of avertin and butanol was injected at 1,200 to 1,500 mg/kg into the abdominal cavity of rats to induce anesthesia of the rats, euthanasia was performed by intracardiac perfusion with 0.9% saline solution to remove blood, and perfusion fixation followed. Then, the knee joint cartilage and bone tissue were extracted, fixed in 10% neutral buffered formalin and 1% CPC (cetylpyridinium chloride) for 1 day, and then decalcified with EDTA (ethylene-diamine tetraacetic acid). After embedding in OCT, tissues was sectioned into the sagittal plane using microtome. OCT-embedded tissue sections were reacted with antibodies of pain-related neuropeptides CGRP, SP, and NK1 receptors as primary antibodies. After secondary antibody reaction, the sections were stained with fluorescent Alexa Fluor dye and observed under a fluorescence microscope.

Immunohistochemistry: A mixture of avertin and butanol was injected at 1,200 to 1,500 mg/kg into the abdominal cavity of rats to induce anesthesia of the rats, euthanasia was performed by intracardiac perfusion with 0.9% saline solution to remove blood, and perfusion fixation followed. The spinal cord (L3-5) at the level of the knee joint was extracted and then post-fixed, and the tissue embedded in OCT was freezing-sectioned and subjected to immunohistochemistry (DAB staining) of CGRP and SP.

Western Blot: A mixture of avertin and butanol was injected at 1,200 to 1,500 mg/kg into the abdominal cavity of rats to induce anesthesia of the rats, euthanasia was performed by intracardiac perfusion with 0.9% saline solution to remove blood, and perfusion fixation followed. The spinal segment (L3-5) dominantly innervating into the knee joint was extracted. After the tissue was disrupted with a protein elution solution, the proteins were separated using SDS-PAGE and transferred to a PVDF membrane. After blocking with 5% blocking buffer, the membrane was reacted with primary antibodies, including NK1 receptor and TRPV1, and inflammation-related factors COX2, IL-1β, and IL-6, and with HRP-attached secondary antibodies. Then, the result was detected by color development with ECL solution and exposed to X-ray films, and the expression level was analyzed using densitometry.

### Example 1

### Confirmation of Effect of GR82334 Administration on Pain Relief

### <1-1> Effect of Intra-Articular Administration of GR82334 on Pain Relief

To confirm the effects of the GR82334 drug on pain relief, 30 μl of GR82334 (10 μM) was injected into the joint cavity in a rat model of MIA-induced chronic pain and analgesic effect was analyzed using paw withdrawal threshold (PWT.

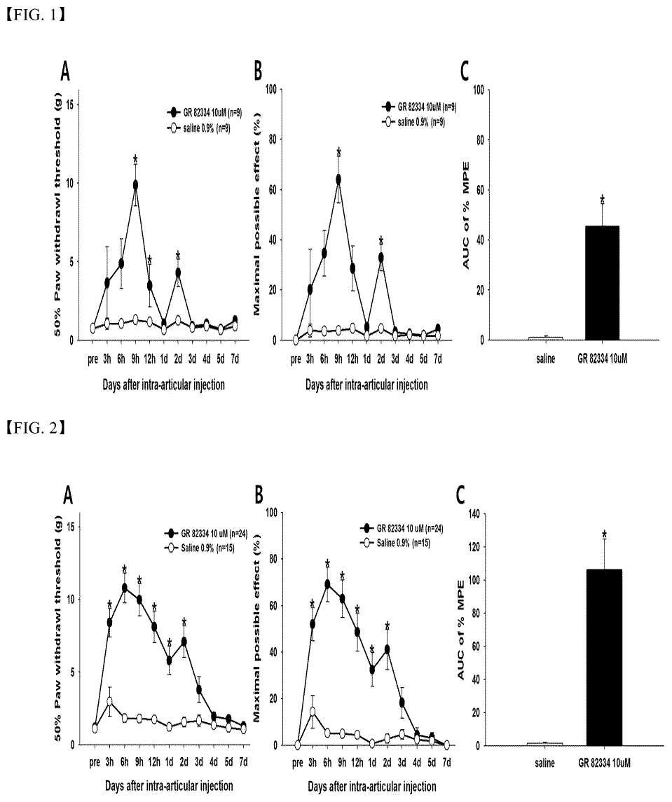

The result of the analysis showed that intra-articular administration of GR82334 (10 μM) significantly reduced pain and, in particular, drastically reduced pain at 9 hours after drug administration. Specifically, MPE (maximal possible effect) indicating pain relief effect as a percentage ratio compared to the pre-value was calculated to be about 59% at 9 hours after administration, which means that the pain is reduced by 59% compared to before drug injection. In addition, intra-articular administration of GR82334 maintains statistically the pharmaceutical analgesic effect for up to 3 days after administration (see FIG. 1).

### <1-2> Effect of Intrathecal Administration of GR82334 on Pain Relief

10 μl of GR82334 (10 μM) was injected intrathecally in a rat model of MIA-induced chronic pain and pain relief effect was analyzed using paw withdrawal threshold (PWT).

The result of the analysis showed that intrathecal administration of GR82334 significantly reduced pain and, in particular, drastically reduced pain at 6 hours after drug administration. Specifically, MPE (maximal possible effect) indicating pain relief effect as a percentage ratio compared to the pre-value was calculated to be about 69% at 6 hours after drug administration, which means that the pain is reduced by 69% compared to before drug injection. In addition, the intrathecal administration of GR82334 statistically maintains the pharmaceutical analgesic effect for up to 3 days after administration (see FIG. 2).

These results show that GR82334 of the present invention has the effect of alleviating pain, and both intra-articular administration and intrathecal administration have the effect of relieving pain. The intrathecal administration exhibits better pain relief effect than the intra-articular administration.

### Example 2

### Pain Relief Effect Depending on Dose of GR82334

The present inventors found through Example 1 that GR82334 has pain-relieving activity and then analyzed the pain-relieving effect depending on the drug dose.

### <2-1> Effect of Intra-Articular Administration of GR82334 on Pain Relief Depending on Dose

30 μl of GR82334 was injected at different concentrations (10 μM, 1 μM, and 100 nM) into the joint cavity in a rat model of MIA-induced chronic pain and pain relief effect was analyzed using paw withdrawal threshold (PWT).

The result of the analysis showed that the pain relief effect increases in proportion to the dose of GR82334. Specifically, when GR82334 was injected at 10 μM, pain was found to be reduced by up to about 59%, and the effect statistically lasted up to 3 days after injection. When GR82334 was injected at 1 μM, pain was reduced by up to about 26% and the effect statistically lasted up to 2 days after injection. Based on these results, the ED50 (effective dose 50) for intra-articular administration of GR82334 was calculated as about 1.32 μM (see FIG. 3).

### <2-2> Effect of Intrathecal Administration of GR82334 on Pain Relief Depending on Dose

10 μl of GR82334 was injected at different concentrations (10 μM, 1 μM, and 100 nM) intrathecally in a rat model of MIA-induced chronic pain and pain relief effect was analyzed using paw withdrawal threshold (PWT).

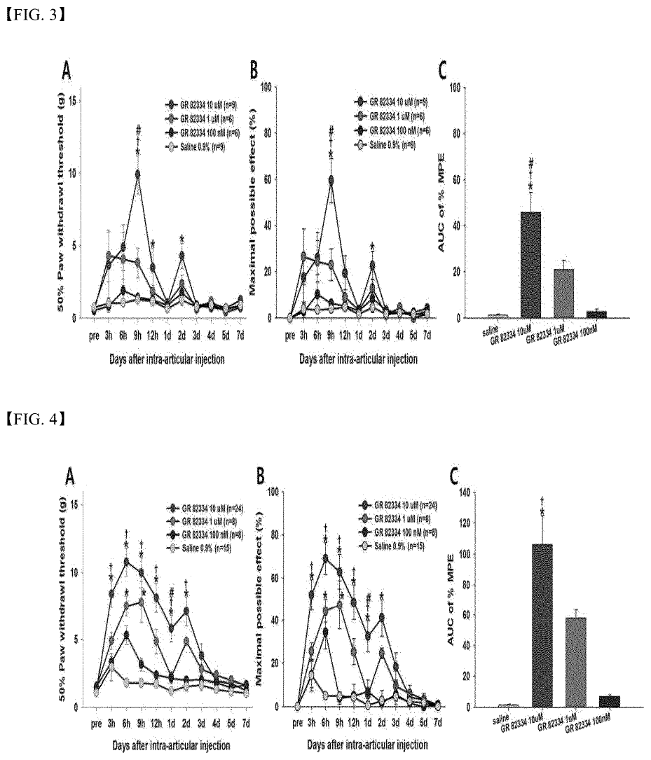

The result of the analysis showed that intrathecal administration increases pain relief effect in proportion to the dose of GR82334. Specifically, when GR82334 was injected at 10 μM, pain was found to be reduced by up to about 69%, and the effect statistically lasted up to 3 days after injection. When GR82334 was injected at 1 μM, pain was reduced by up to about 47% and the effect statistically lasted up to 3 days after injection. Based on these results, the ED50 (effective dose 50) for intrathecal administration of GR82334 was calculated as about 0.96 μM (see FIG. 4).

Based on these results, the present inventors found that intrathecal administration of GR82334 exhibits better pain relief effect at a lower concentration compared to intra-articular administration, and that intrathecal administration is more effective in relieving pain than intra-articular administration.

### Example 3

### Analysis of Expression Changes of Pain-Related Molecules in Subchondral Bone after GR82334 Administration

Based on experiments in the previous examples, the present inventors found that GR82334 has an effect on alleviating pain. Accordingly, the present inventors determined whether GR82334 could regulate the expression of pain-related neuropeptides, such as CGRP (calcitonin gene-related peptide) and SP (substance P), and NK1 receptors, in pain signaling pathways.

For this purpose, GR82334 was injected intra-articularly or intrathecally to rats with chronic pain induced by MIA, and the expression levels of CGRP, SP, and NK1 receptors in the subchondral bone of the knee joint were analyzed. At this time, as a control group, a group treated with saline was used instead of GR82334.

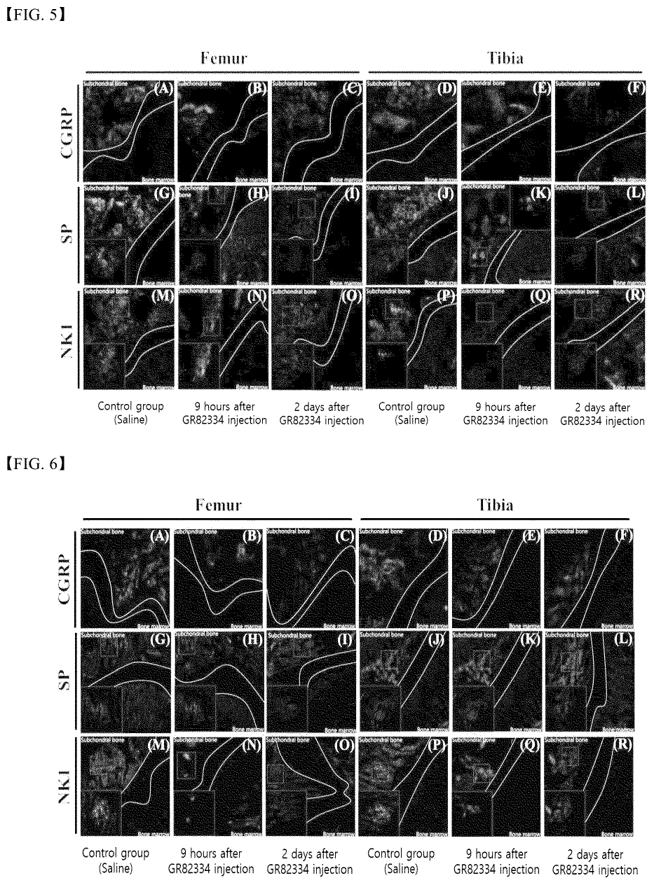

As shown in the results of immunofluorescence (FIGS. 5 and 6), the expression levels of both CGRP and SP decreased in the subchondral bone of both groups intra-articularly and intrathecally administered GR82334, compared to the control group.

In addition, compared to the control group, the GR82334-administered intra-articularly group exhibited an increase in expression of NK1 receptor in the subchondral bone of joint joint at 9 hours after administration, and a decrease in the expression at 2 days after administration. Meanwhile, the intrathecal administration group exhibited a decrease in expression of NK1 receptor at both 9 hours and 2 days after GR82334 administration.

Based on these results, the present inventors found that GR82334 has effects on relieving pain, through both intra-articular and intrathecal administration reduced the expression of molecules involved in pain transmission in the subchondral bone in the knee joint of rats administered GR82334.

### Example 4

### Analysis of Expression Changes of Pain-Related Molecules and Inflammatory Factors in Spinal Cord after GR82334 Administration

GR82334 was injected intra-articularly or intrathecally to rats with MIA-induced chronic pain. At 9 hours and 2 days after injection, changes in the expression of pain-related neuropeptides, such as CGRP and SP, were analyzed in the spinal dorsal horn using immunohistochemistry.

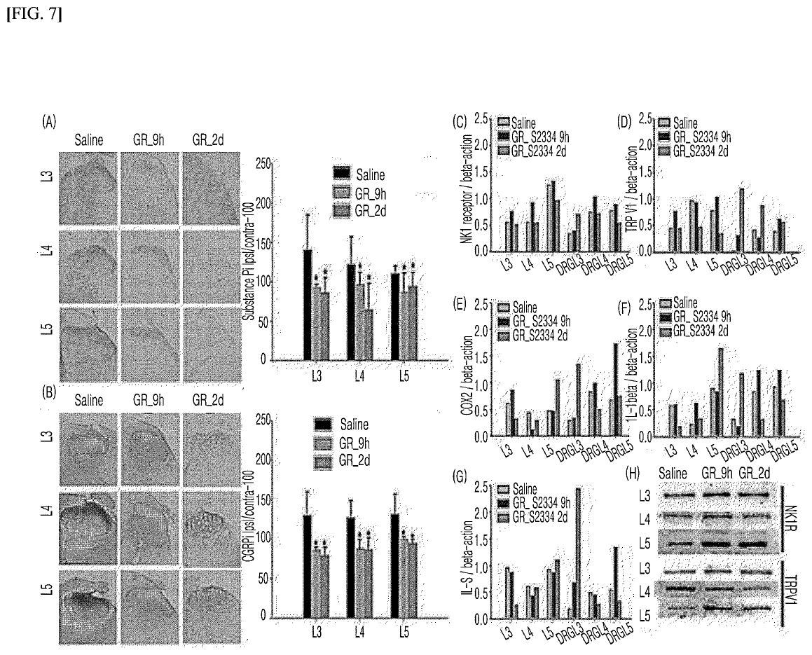

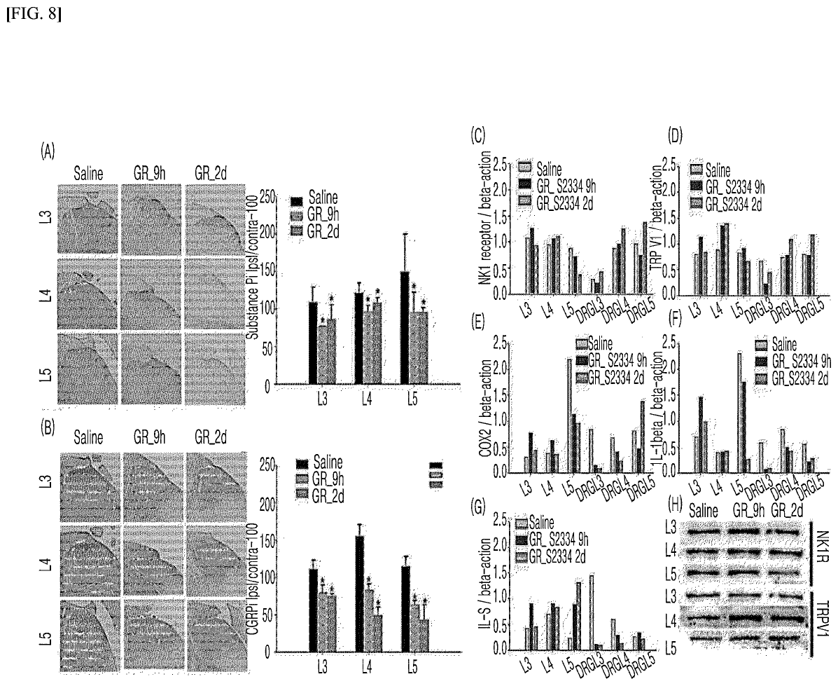

The result shows that the expression of CGRP and SP in the substantia gelatinosa (larmina I, II) of the spinal dorsal horn of rats both intra-articularly and intrathecally administered GR82334 decreased, compared to the control group (FIG. 7A-7B, and FIG. 8A-8B).

Therefore, these results showed that intra-articular and intrathecal administration of GR82334 reduces the expression of pain-related molecules in the spinal doral horn, thereby alleviating pain.

Furthermore, the present inventors examined the expression of extracellular NK-1 and TRPV1 receptors, as pain-transmitting receptors, COX2 and IL-6 as inflammatory cytokines, and IL-6 (interlukin-6) as anti-inflammatory cytokine in the ipsilateral dorsal quadrants of the spinal segment by Western blot at 9 hours and 2 days after intra-articular or intrathecal administration of GR82334.

As shown in FIG. 7C-H and FIG. 8C-H, the results show that extracellular NK1 receptor expression was increased in the ipsilateral dorsal quadrants of spinal segment of rats both intra-articularly and intrathecally administrated GR82334, compared to the control group, which indicates that NK1 receptor antagonist administered binds to the NK1 receptor and prevents internalization of the NK1 receptor into the cells for pain signal transmission, thereby reducing SP-NK1 intracellular signaling by intra-articular and intrathecal administration of the NK1 receptor antagonist.

In addition, the expression of IL-6, an anti-inflammatory cytokine, was found to increase in the ipsilateral dorsal quadrants of spinal segment of rats both intra-articularly and intrathecally administrated GR82334, compared to the control group.

Based on these results, the present inventors found that GR82334 can reduce pain by controlling pain-transmitting signals, and as well as relieve inflammation due to anti-inflammatory activity.

Hereinbefore, the present invention has been described with reference to preferred embodiments. Those skilled in the field to which the present invention pertains will appreciate that the present invention may be applied in modified forms without departing from the essential characteristics of the present invention. Therefore, the disclosed embodiments should be considered from an illustrative point of view, not from a restrictive point of view. The scope of the present invention is defined by the claims rather than the foregoing description and all differences equivalent thereto should be construed as falling into the scope of the present invention.

1. A pharmaceutical composition for preventing or treating pain comprising GR82334 having the following formula as an active ingredient:

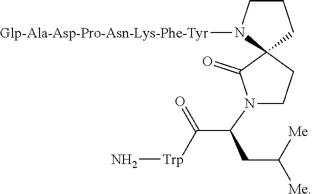

2. The pharmaceutical composition according to claim 1, wherein the composition is administered intra-articularly or intrathecally.

3. The pharmaceutical composition according to claim 1, wherein the pain is chronic pain.

4. The pharmaceutical composition according to claim 3, wherein the chronic pain is selected from the group consisting of neuropathic pain, complex regional pain syndrome, postherpetic neuralgia, and pain syndrome after spinal surgery.

5. The pharmaceutical composition according to claim 4, wherein the neuropathic pain is induced by at least one nerve damage selected from the group consisting of peripheral nerve damage, central nerve damage, and inflammatory nerve damage.

6. The pharmaceutical composition according to claim 4, wherein the neuropathic pain is selected from the group consisting of multiple sclerosis including abnormalities of or damage to the peripheral nervous system and spinal nerve damage, spinal cord injury, hyperalgesia, hyperesthesia, neuropathy, diabetic neuropathy, neuritis, neuralgia, burning pain, allodynia, postherpetic neuralgia, lumbar nerve root compression, cancer-related pain, pain due to alcoholism, atypical facial pain, herpetic neuralgia, poststroke pain, HIV-related neuropathy, osteoarthritis pain, joint pain, and phantom limb pain.

7. The pharmaceutical composition according to claim 1, wherein the GR82334 relieves or reduces pain through activity of blocking the SP (substance P)/NK1 (neurokinin 1) signaling pathway.

8. The pharmaceutical composition according to claim 1, wherein the GR82334 reduces expression of an inflammatory cytokine and increases expression of an anti-inflammatory cytokine.

9. The pharmaceutical composition according to claim 8, wherein the inflammatory cytokine comprises COX-2and IL-1β, and the anti-inflammatory cytokine comprises IL-6.

10. A method of treating pain comprising intra-articularly or intrathecally administering the pharmaceutical composition according to claim 1 to a subject suffering from pain.
